SEQUENCE LISTING (4 sequences)

SEQ ID NO: 1 - DNA, 182 bp, Artificial Sequence
  Synthetic construct codes for single chain peptide with restriction enzyme sites EcoRI and NotI included. Ochre stop codon closes reading frame.
  CDS (7)..(171)
gaattcttgccagagactggtgtcaagcagcacttgtgtggttctcatttggttgaggccttgtacttggtttgtggtgagagaggtttcttctacctgccagaaaccggtatcgttgagcagtgttgtacttccatctgttccctgtaccagttggagaactactgtaactaagcggccgc

SEQ ID NO: 2 - protein, 55 aa, Artificial Sequence
  The expressed single chain peptide, does not include result of fusion to MAT-alpha secretion factor via EcoRI
LPETGVKQHLCGSHLVEALYLVCGERGFFYLPETGIVEQCCTSICSLYQLENYCN

SEQ ID NO: 3 - protein, 21 aa, Homo sapiens
GIVEQCCTSICSLYQLENYCN

SEQ ID NO: 4 - protein, 30 aa, Homo sapiens
FVNQHLCGSHLVEALYLVCGERGFFYTPKT

(19) United States
(12) Patent Application Publication
(10) Pub. No.: US 2022/0324937 A1
(43) Pub. Date: Oct. 13, 2022

(54) METHODS FOR EXPRESSING HUMAN INSULIN DERIVATIVE WITH A TERMINAL LPXT THEREOF

(71) Applicant: Arri Russell Morris, Austin, TX (US)

(72) Inventor: Arri Russell Morris, Austin, TX (US)

(21) Appl. No.: 17/715,309

(22) Filed: Apr. 7, 2022

Related U.S. Application Data
(63) Continuation-in-part of application No. 17/677,670, filed on Feb. 22, 2022.
Application No. 17/677,670 is a continuation of application No. 16/719,788, filed on Dec. 18, 2019, now Pat. No. 11,261,228.

(51) Int. Cl. C07K 14/62; C12P 21/02

(52) U.S. Cl. CPC C07K 14/62; C12P 21/02

(57) ABSTRACT

The present disclosure relates to methods for expressing human insulin derivative in which the motif Leucine-Proline-X-Threonine appears at end of B-chain, where X is any amino acid residue. Said human insulin derivative is expressed using insulin derivative peptide and sortase. The method is to translate a nucleotide sequence encoding a single chain peptide into a precursor of human insulin derivative and combine the single chain peptide with sortase to convert a human insulin derivative precursor into a fully folded, biologically active human insulin derivative.

### REFERENCE TO SEQUENCE LISTING SUBMITTED ELECTRONICALLY

This application contains a sequence listing, which is submitted electronically via EFS-Web as an ASCII formatted sequence listing with a file name “Sequence_Listing.txt”, creation date of Apr. 7, 2022, and having a size of 3 KB. The sequence listing submitted via EFS-Web is part of the specification and is herein incorporated by reference in its entirety.

### CROSS REFERENCE TO RELATED APPLICATIONS

The present application is a continuation-in-part of U.S. patent application Ser. No. 17/677,670, filed Feb. 22, 2022, which is a continuation of U.S. patent application Ser. No. 16/719,788, filed Dec. 18, 2019 (now U.S. Pat. No. 11,261,228); all of which are incorporated by reference herein.

### TECHNICAL FIELD

The disclosure relates to a process of expressing a fully folded functional two chain insulin derivate that requires no further processing to make it functionally active, using an insulin derivative peptide and sortase.

### BACKGROUND

The incidence of diabetes mellitus has risen from approximately 108 million patients in 1980 to an estimated 422 million in 2014. Meanwhile, cost-related insulin underuse is reported to affect one in four American patients with diabetes. The concurrent advent of insulin rationing-related deaths calls for new, cost-effective approaches to the synthesis of insulin derivatives.

One such derivative is insulin glulisine expressed in a host organism, Escherichia coli. The commercial manufacturing process for the insulin glulisine requires 15 steps that broadly fall under the categories including cell culture and harvest, downstream processing, and final purification. In summary, the insulin glulisine fusion protein is expressed by Escherichia coli and stored in inclusion bodies within the E. coli cells. The fusion protein is folded and then enzymatically converted during downstream processing. The product of the downstream processing is then purified by chromatography, usually at a separate facility.

U.S. Pat. No. 6,221,633B1 describes insulin glulisine and alternative fast-acting insulin compounds. The limitation of this prior art is that multiple proinsulin plasmids must be expressed, stringing together several peptides to form the final insulin protein. US2015/0118710A1 describes a process in which the yeast-derived Kexin enzyme creates insulin glargine from a single peptide precursor. However, the limitation of this prior art is that the B-chain must end with two Arginine amino acid residues. Recognizing this limitation, the claims of US2015/0118710A1 were limited to the long-acting insulin glargine.

### SUMMARY

The present disclosure relates to a process of expressing a fully folded functional two chain insulin derivate. Said process comprises steps of:
i) translating a nucleotide sequence encoding a single chain peptide into a precursor of an insulin derivative;
ii) combining the single chain peptide with sortase to convert the insulin derivative precursor into a fully folded, biologically active insulin derivative.

In an embodiment of the proposed disclosure, the process would yield a human insulin derivative in which the A-Chain is SEQ ID No.3 and the B-chain is modified version of SEQ ID No.4, in which position 1 is substituted with glycine, position 3 is substituted with lysine, position 27 is substituted with leucine and position 29 is substituted with glutamic acid.

In an embodiment of the proposed disclosure, the gene encoding single chain insulin derivative peptide is set forth as SEQ ID No. 1, or the expressed peptide contains SEQ ID No. 2 starting from the fourth amino acid residue.

In an embodiment of the proposed disclosure, the sortase gene is derived from one of bacteria of order Lactobacillales, or an organism genetically modified to contain the genetic sequence of a Lactobacillales bacteria.

In an embodiment of the proposed disclosure, the sortase is expressed extracellularly by one host organism, and the single chain peptide is expressed separately by another host organism of the same species, or of a different species.

In an embodiment of the proposed disclosure, the human insulin derivative or a physiologically tolerable salt thereof, is indicated as the Formula I shown below.

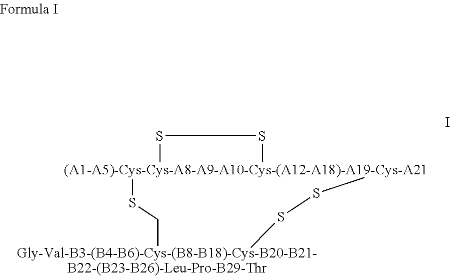

in which
(A1-A5) are the amino acid residues in the positions A1 to A5 of the A chain of human insulin or animal insulin,
(A12-A17) are the amino acid residues in the positions A12 to A17 of the A chain of human insulin or animal insulin, A8 and A10 are the amino acid residues in positions A8 and A10 of the A chain of human insulin or animal insulin, A9 is a serine residue (Ser) or alanine residue (Ala), A19 is Tyr, Phe or Ser, A21 is Asn, Asp, Gly, Ser, Thr or Ala,
(B4-B6) are the amino acid residues in the positions B4 B6 of the B chain of human insulin or animal insulin,
(B8-B18) are the amino acid residues in the positions B8 B18 of the B chain of human insulin or animal insulin,
(B23-B26) are the amino acid residues in the positions B23 to B26 of the B chain of human insulin or animal insulin,
B3 is Arg, Lys, His, Ala or Asn,
B20 is a glycine residue (Gly) or alanine residue (Ala),
B21 is a glutamic acid residue (Glu) or alanine residue (Ala),
B22 is an arginine residue (Arg) or alanine residue (Ala),
B29 is any naturally occurring amino acid residue.

### BRIEF DESCRIPTION OF DRAWINGS

FIG. 1 shows a modified pPIC9K vector with modifications to initial pPIC9K vector for constitutive expression of sortase;

FIG. 2 shows a modified pGAPZα vector with modifications to the initial pGAPZα vector for constitutive expression of the single chain peptide human/animal insulin derivative precursor;

FIG. 3 shows a cleavage site illustrating sites of cleavage for the proposed human insulin derivative precursor and the resulting folded structure; and

FIG. 4 shows Formula I of the proposed human insulin derivative illustrating relative positions and bonds of amino acid residues.

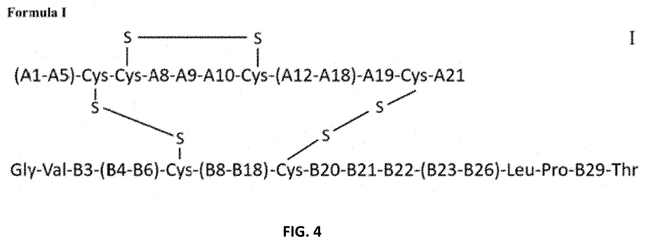

### DETAILED DESCRIPTION

The features of the present disclosure will become more fully apparent from the following description taken in conjunction with the accompanying drawings. Understanding that the drawings depict only several embodiments in accordance with the disclosure and are therefore, not to be considered limiting of its scope, the disclosure will be described with additional specificity and detail through use of the accompanying drawings and sequence listing comprising:

SEQ ID No.1 representing the systematic gene designed to express a single chain peptide.

SEQ ID No.2 representing the expressed single chain peptide.

SEQ ID No.3 representing the A chain of human insulin.

SEQ ID No.4 representing the B chain human insulin.

Human insulin derivatives are derivatives of naturally occurring insulins, namely human insulin (SEQ ID No. 3 illustrates A-chain of human insulin; see SEQ ID No. 4 illustrates B-chain of human insulin, sequence listing) or animal insulins which differ from the corresponding, otherwise identical naturally occurring human insulin by substitution of at least one naturally occurring amino acid residue and/or addition of at least one amino acid residue and/or organic residue.

Of the twenty naturally occurring amino acids which are universally encodable, the amino acids glycine (Gly), alanine (Ala), Valine (Val), leucine (Leu), isoleucine (Ile), Serine (Ser), threonine (Thr), cysteine (Cys), methionine (Met), asparagine (ASn), glutamine (Gin), phenylalanine (Phe), tyrosine (Tyr), tryptophan (Trp) and proline (Pro) are designated here as neutral amino acids, the amino acids arginine (Arg), lysine (LyS) and histidine (His) are designated as basic amino acids and the amino acids aspartic acid (Asp) and glutamic acid (Glu) are designated as acidic amino acids.

The present disclosure relates to insulin derivatives which, in comparison to current derivatives, have a simplified process for synthesis that relies heavily on recombinant DNA technology. Although there are many novel insulin derivatives that could be created in a number of hosts by following conventional processes, the present disclosure focuses on a derivative similar to insulin glulisine expressed in a yeast host organism, Pichia pastoris. In the proposed disclosure, the method to concurrently express the sortase from the bacteria Staphylococcus aureus, and a single chain peptide containing the sequence Leucine-Proline-X-Threonine is disclosed in the present disclosure. X is any amino acid residue.

The proposed process of expressing a fully folded functional two chain human insulin derivate requires no further processing to make it functionally active. The novel use of sortase from Staphylococcus aureus eliminates the need for downstream processing, can be performed at a single facility, and reduces expenditure for catalysts.

The process of expressing the fully folded functional two chain insulin derivate includes to clone an insulin derivative peptide and the sortase in a yeast. In an embodiment, the yeast may be Pichia pastoris or a bacterium, like Escherichia coli. The sequence coding for the sortase is put under the control of constitutive promoter or inducible promoter. The gene encoding single chain insulin derivative peptide is set forth as SEQ ID No. 1, or the expressed peptide contains SEQ ID No. 2 starting from the fourth amino acid residue. The sortase gene is derived from one of bacteria f order Lactobacillales, and an organism genetically modified to contain the genetic sequence of a Lactobacillales bacteria.

Further, the said peptide and sortase are co-expressed. As used herein the term “expression” refers to a process by which a peptide is produced based on the nucleic acid sequence of a gene. The process includes both transcription and translation. The sortase is expressed extracellularly by one host organism, and the single chain peptide is expressed separately by another host organism of the same species, or of a different species. By co-expressing, the peptide and the sortase, a fully functional insulin derivative with Leucine-Proline-X-Threonine motif at the terminal of the B chain, where X is any amino acid residue, is obtained. Considering, A-Chain is SEQ ID No.3, and the B-chain is modified version of SEQ ID No.4. Position 1 is substituted with glycine, position 3 is substituted with lysine, position 27 is substituted with leucine and position 29 is substituted with glutamic acid, to obtain the fully functional insulin derivative.

This process trivializes the effort required to create new human insulin derivatives as no intermediate catalysts or processes would need to be developed. As the limitations laid out in example 3 only differ from human insulin by two amino acid residues at positions B1 and B27, this method would allow for rapid prototyping of insulin derivatives in parallel cell lines. Once the right formulation is experimentally determined, a different method of manufacturing could be devised.

Because Pichia pastoris has recently received recognition for efficient recombinant expression of proteins, the first example will point to a practical approach of making the two modifications on the same host organism. However, it is possible that multiple host organisms could achieve the same result, if incubated separately.

In another embodiment, the process of converting a single chain peptide into fully folded biologically active insulin derivative is disclosed. Initially, nucleotide sequence encoding a single chain peptide is translated into a precursor of an insulin derivative. Further, the single chain peptide is combined with sortase to convert the human insulin derivative precursor into a fully folded, biologically active human insulin derivative.

The proposed human insulin derivative or the physiologically tolerable salt is indicated as Formula 1, shown below:

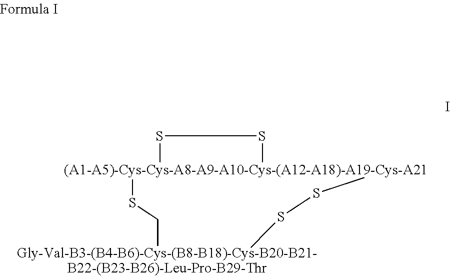

in which
(A1-A5) are the amino acid residues in the positions A1 to A5 of the A chain of human insulin or animal insulin,
(A12-A17) are the amino acid residues in the positions A12 to A17 of the A chain of human insulin or animal insulin,
A8 and A10 are the amino acid residues in positions A8 and A10 of the A chain of human insulin or animal insulin,
A9 is a serine residue (Ser) or alanine residue (Ala),
A19 is Tyr, Phe or Ser,
A21 is Asn, Asp, Gly, Ser, Thr or Ala,
(B4-B6) are the amino acid residues in the positions B4 to B6 of the B chain of human insulin or animal insulin,
(B8-B18) are the amino acid residues in the positions B8 to B18 of the B chain of human insulin or animal insulin,
(B23-B26) are the amino acid residues in the positions B23 to B26 of the B chain of human insulin or animal insulin,
B3 is Arg, Lys, His, Ala or Asn,
B20 is a glycine residue (Gly) or alanine residue (Ala),
B21 is a glutamic acid residue (Glu) or alanine residue (Ala),
B22 is an arginine residue (Arg) or alanine residue (Ala),
B29 is any naturally occurring amino acid residue.

### Illustrative Example 1

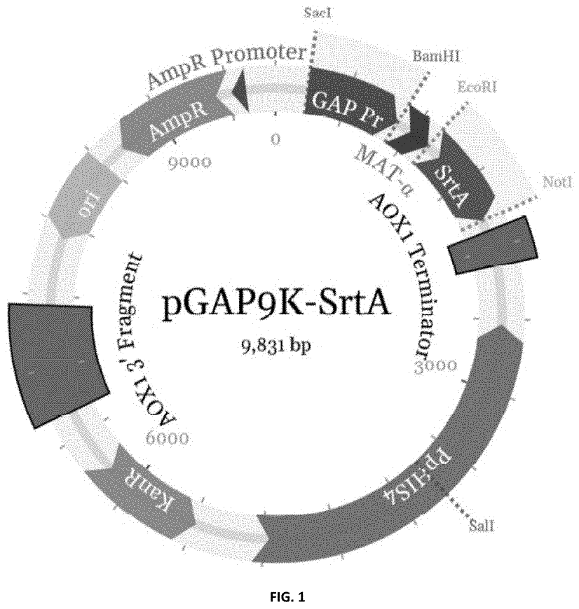

Following the procedure laid out by Zhao et al. published in March of 2017, the expression of sortase is achieved extracellularly by modifying the commercially available pPIC9K vector to use the GAP Promoter by using the restriction enzyme sites SacI and BamHI. Then, using the genetic material of Staphylococcus aureus from cell line ATCC 35556, the gene encoding sortase (SrtA) is inserted to the modified pPIC9K vector with the restriction enzymes EcoRI and NotI. The modified vector is linearized by the SalI restriction enzyme to increase transformant expression of sortase in the yeast, Pichia pastoris. The resulting plasmid is shown in FIG. 1. This modification is selected by growing the yeast on petri dishes with Zeocin agar.

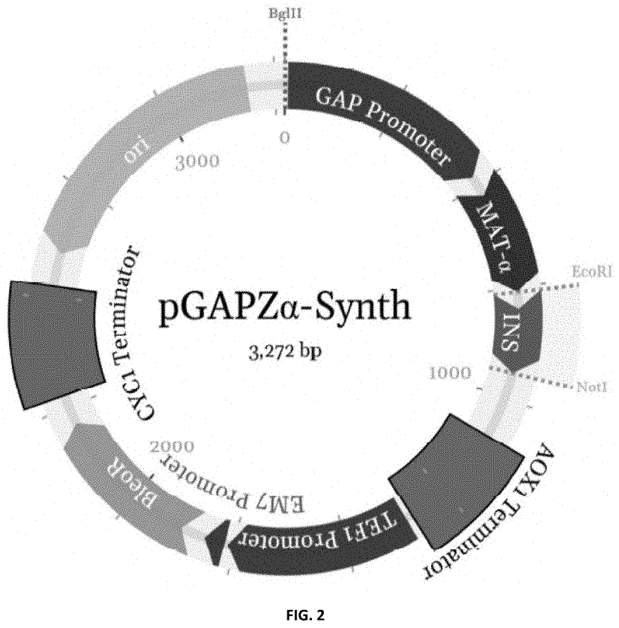

Next, the commercially available pGAPZα vector can be used on the selected transformant Pichia pastoris cell line. SEQ ID No. 1 is prepared synthetically and sent in a plasmid. Once the sequence is isolated from the plasmid with the restriction enzymes EcoRI and NotI, it can be inserted in pGAPZα as shown in FIG. 2. This time, the resulting plasmid is linearized by BglII to increase expression efficiency and transformant cells are selected with Geneticin.

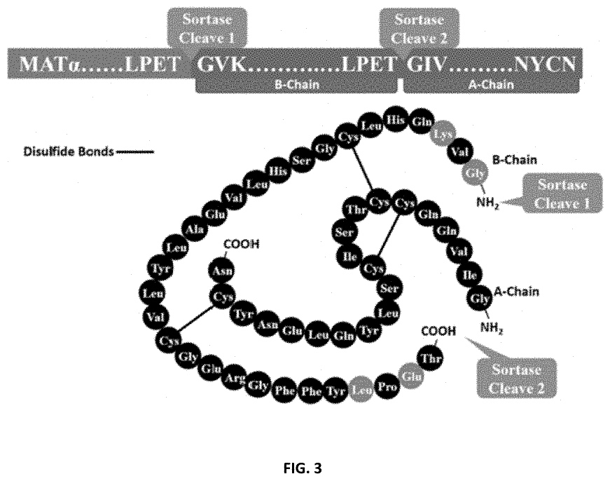

When the final Pichia pastoris transformant cell line sufficiently expresses alpha secretion factor with the single chain peptide from SEQ ID No. 2, sortase cleaves the single chain peptide as shown in FIG. 3. In FIG. 3, the differences between the resulting insulin derivative and human insulin are highlighted as gray amino acid residues.

### Illustrative Example 2

Another embodiment of the disclosed invention would be to express sortase using Escherichia coli following a procedure similar to the 2017 Wu et al. article provided as a supplementary reference. Then, the vector from FIG. 2 could be used to express the precursor peptide in Pichia pastoris. The resulting products would need to be extracted and combined as an intermediate step prior to final filtration. Although this is, in some ways, less efficient than example 1, it could be significantly more cost effective than the current process for similar insulin derivatives.

### Illustrative Example 3

The limitation imposed on the formulation of insulin derivatives by the process of preceding examples is that each chain of insulin must begin with Glycine, and the B-chain must end with Leucine-Proline-X-Threonine, where X is any amino acid. While this limitation is unique and specific, there are thousands of potentially viable insulin derivatives. When efficient expression of sortase is achieved, as in the first paragraph of example 1, the single chain peptide of SEQ ID No. 2 could arbitrarily be manipulated at relatively low cost by inserting a marginally different sequence into the commercial pGAPZα vector prior to linearization.

1. An interbody spinal implant for insertion between adjacent vertebral bodies of a human spine, said implant comprising:
a leading end for introduction of said spinal implant into the spine, an opposite trailing end, spaced apart sides therebetween, and a mid-longitudinal axis passing through said leading and trailing ends;
opposite upper and lower surfaces between said leading and trailing ends and said spaced apart sides, said upper surface adapted for placement in engagement with the bone of one of the vertebral bodies and said opposite lower surface adapted for placement in engagement with the bone of the other of the vertebral bodies when said implant is placed between the adjacent vertebral bodies; and
a plurality of surface projections formed on said upper and lower surfaces of said implant, at least one of said surface projections having a forward facet directed at least in part toward the leading end, a rearward facet directed at least in part toward the trailing end, and opposed side facets converging toward one another between said forward facet and said rearward facet, said forward facet and said rearward facet having a length and a slope, the length of said forward facet being longer than the length of said rearward facet, the slopes of said forward facet and said rearward facet both being in a negative direction of inclination, said at least one of said surface projections having a base between said forward and rearward facets and between said opposed side facets, said base being in the shape of a rectangle, said base having a width and a length greater than the width, the length extending along the mid-longitudinal axis of said implant, the width being transverse to the length.

2. The spinal implant of claim 1, wherein one of said facets is formed in the shape of a triangle having a vertex with an included angle greater than 90 degrees.

3. The spinal implant of claim 1, wherein the slope of said rearward facet is steeper than the slope of said forward facet.

4. The spinal implant of claim 1, wherein said rearward facet is at an angle to at least one of said upper and lower surfaces of said implant.

5. The spinal implant of claim 4, wherein said angle is greater than 90 degrees.

6. The spinal implant of claim 1, wherein said surface projections are oriented relative to one another to form an array.

7. (canceled)

8. The spinal implant of claim 1, wherein said upper and lower surfaces of said implant are at least in part arcuate.

9. The spinal implant of claim 1, wherein at least one of said leading end, trailing end, and sides are curved.

10. The spinal implant of claim 1, wherein said sides are curved.

11. The spinal implant of claim 1, wherein each of said leading end, trailing end, and sides are curved.

12. The spinal implant of claim 11, wherein said leading end, trailing end, and sides form a circle.

13. The spinal implant of claim 1, wherein said upper and lower surfaces of said implant are at least in part planar.

14. The spinal implant of claim 1, wherein said upper and lower surfaces converge toward each other along at least a portion of the length of said implant.

15. The spinal implant of claim 1, wherein said implant comprises a material other than bone.

16. The spinal implant of claim 1, wherein said implant comprises bone.

17. The spinal implant of claim 16, wherein said bone includes cortical bone.

18. The spinal implant of claim 1, wherein said implant comprises bone growth promoting material.

19. The spinal implant of claim 18, wherein said bone growth promoting material is one of bone morphogenetic protein, hydroxyapatite, and genes coding for the production of bone.

20. The spinal implant of claim 1, wherein said implant is treated with a bone growth promoting substance.

21. The spinal implant of claim 1, wherein said implant is a source of osteogenesis.

22. The spinal implant of claim 1, wherein said implant is at least in part bioabsorbable.

23. The spinal implant of claim 1, wherein said implant comprises metal.

24. (canceled)

25. The spinal implant of claim 23, wherein said metal includes titanium.

26. The spinal implant of claim 1, wherein said implant comprises at least one of a plastic material, a ceramic material, and a porous material.

27-28. (canceled)

29. The spinal implant of claim 1, wherein said implant is formed of a material that intrinsically participates in the growth of bone from one of the adjacent vertebral bodies to the other of the adjacent vertebral bodies.

30. The spinal implant of claim 1, wherein said implant is a motion preserving device adapted to space apart and allow motion between the adjacent vertebral bodies.

31. The spinal implant of claim 1, wherein said spinal implant is a fusion implant.

32. The spinal implant of claim 31, wherein said upper and lower surfaces include at least one opening to permit bone growth from adjacent vertebral body to adjacent vertebral body through said implant.

33. The spinal implant of claim 31, wherein said implant has an internal chamber and an access opening for accessing said internal chamber.

34. The spinal implant of claim 33, wherein said implant has a cap for closing said access opening.

35. The spinal implant of claim 33, wherein said upper and lower surfaces include at least one opening in communication with said internal chamber to permit bone growth from adjacent vertebral body to adjacent vertebral body through said implant.

36. The spinal implant of claim 33, wherein said internal chamber is capable of containing bone growth promoting material.

37. The spinal implant of claim 36, further comprising said bone growth promoting material, said bone growth promoting material being one of bone morphogenetic protein, hydroxyapatite, and genes coding for the production of bone.

38. The spinal implant of claim 1, further comprising at least one opening capable of retaining fusion promoting materials.

39. The spinal implant of claim 1, in combination with a fusion promoting substance.

40. The spinal implant of claim 39, wherein said fusion promoting substance includes at least one of bone, bone morphogenetic protein, hydroxyapatite, and genes coding for the production of bone.

41. The spinal implant of claim 1, wherein each of said surface projections have a base, at least two of said bases being adjacent to one another.

42. The spinal implant of claim 1, wherein each of said surface projections have a base, at least two of said bases being spaced apart from one another along a direction generally parallel to the mid-longitudinal axis of said implant.

43. The spinal implant of claim 1, wherein each of said surface projections have a base, at least two of said bases being spaced apart from one another along a direction generally transverse to the mid-longitudinal axis of said implant.

44. The spinal implant of claim 1, wherein another of said surface projections has a forward facet, said forward facets of said surface projections facing the same direction.

45. The spinal implant of claim 1, wherein said opposed side facets are directed generally toward said spaced apart sides of said implant, respectively, said side facets converging toward each other in a direction away from one of said upper and lower surfaces of said implant.

46. The spinal implant of claim 1, wherein said opposed side facets intersect each other.

47. The spinal implant of claim 46, wherein said opposed side facets converge to form a peak at the top of said surface projection.

48. The spinal implant of claim 47, wherein said peaks of at least two of said surface projections are aligned along lines that are at least one of perpendicular, parallel, and diagonal to the mid-longitudinal axis of said implant.

49. The spinal implant of claim 47, wherein said peak of one of said surface projections overlies at least a portion of another of said surface projections.

50. The spinal implant of claim 47, wherein said peaks of at least two of said surface projections are at the same height above one of said upper and lower surfaces of said implant.

51. The spinal implant of claim 45, wherein adjacent side facets of adjacent surface projections are spaced apart to define a groove therebetween.

52. The spinal implant of claim 45, wherein a plurality of adjacent surface projections are spaced apart to form a plurality of grooves therebetween.

53. The spinal implant of claim 52, wherein at least one of said grooves is parallel to the mid-longitudinal axis of said implant.

54. The spinal implant of claim 52, wherein at least two of said grooves cross each other.

55. The spinal implant of claim 52, wherein at least one of said grooves has a horizontal cross-sectional shape that is one of a v-shape, u-shape, and a box like shape.

56. (canceled)

57. An interbody spinal implant for insertion between adjacent vertebral bodies of a human spine, said implant comprising:
a leading end for introduction of said spinal implant into the spine, an opposite trailing end, spaced apart sides therebetween, and a mid-longitudinal axis passing through said leading and trailing ends;
opposite upper and lower surfaces between said leading and trailing ends and said spaced apart sides, said upper surface adapted for placement in engagement with the bone of one of the vertebral bodies and said opposite lower surface adapted for placement in engagement with the bone of the other of the vertebral bodies when said implant is placed between the adjacent vertebral bodies; and
a plurality of surface projections formed on said upper and lower surfaces of said implant, at least a first and a second of said surface projections each having at least one forward facing facet directed at least in part toward said leading end, at least one rearward facet directed at least in part toward said trailing end, and opposed side facets converging toward one another between said forward facet and said rearward facet, each of said forward facet and rearward facet having a length and a slope, the length of said forward facet being longer than the length of said rearward facet, the slope of said rearward facet being steeper than the slope of said forward facet, said first and second surface projections each forming a base and a portion above said base, said portion above the base of said first and second surface projections extending outside of the perimeter of the base of a respective one of said first and second surface projections, said at least one of said surface projections having a base between said forward and rearward facets and between said opposed side facets, said base being in the shape of a rectangle, said base having a width and a length greater than the width, the length extending along the mid-longitudinal axis of said implant, the width being transverse to the length.

58. The spinal implant of claim 57, wherein said rearward facet is at an angle to at least one of said upper and lower surfaces of said implant.

59. The spinal implant of claim 58, wherein said angle is greater than 90 degrees.

60. The spinal implant of claim 57, wherein said surface projections are oriented relative to one another to form an array.

61. (canceled)

62. The spinal implant of claim 57, wherein said upper and lower surfaces of said implant are at least in part arcuate.

63. The spinal implant of claim 57, wherein at least one of said leading end, trailing end, and sides are curved.

64. The spinal implant of claim 57, wherein said sides are curved.

65. The spinal implant of claim 57, wherein each of said leading end, trailing end, and sides are curved.

66. The spinal implant of claim 65, wherein said leading end, trailing end, and sides form a circle.

67. The spinal implant of claim 57, wherein said upper and lower surfaces of said implant are at least in part planar.

68. The spinal implant of claim 57, wherein said upper and lower surfaces converge toward each other along at least a portion of the length of said implant.

69. The spinal implant of claim 57, wherein said implant comprises a material other than bone.

70. The spinal implant of claim 57, wherein said implant comprises bone.

71. The spinal implant of claim 70, wherein said bone includes cortical bone.

72. The spinal implant of claim 57, wherein said implant comprises bone growth promoting material.

73. The spinal implant of claim 72, wherein said bone growth promoting material is one of bone morphogenetic protein, hydroxyapatite, and genes coding for the production of bone.

74. The spinal implant of claim 57, wherein said implant is treated with a bone growth promoting substance.

75. The spinal implant of claim 57, wherein said implant is a source of osteogenesis.

76. The spinal implant of claim 57, wherein said implant is at least in part bioabsorbable.

77. The spinal implant of claim 57, wherein said implant comprises metal.

78. (canceled)

79. The spinal implant of claim 77, wherein said metal includes titanium.

80. The spinal implant of claim 57, wherein said implant comprises at least one of a plastic material, a ceramic material, and a porous material.

81-82. (canceled)

83. The spinal implant of claim 57, wherein said implant is formed of a material that intrinsically participates in the growth of bone from one of the adjacent vertebral bodies to the other of the adjacent vertebral bodies.

84. The spinal implant of claim 57, wherein said implant is a motion preserving device adapted to space apart and allow motion between the adjacent vertebral bodies.

85. The spinal implant of claim 57, wherein said spinal implant is a fusion implant.

86. The spinal implant of claim 85, wherein said upper and lower surfaces include at least one opening to permit bone growth from adjacent vertebral body to adjacent vertebral body through said implant.

87. The spinal implant of claim 85, wherein said implant has an internal chamber and an access opening for accessing said internal chamber.

88. The spinal implant of claim 87, wherein said implant has a cap for closing said access opening.

89. The spinal implant of claim 87, wherein said upper and lower surfaces include at least one opening in communication with said internal chamber to permit bone growth from adjacent vertebral body to adjacent vertebral body through said implant.

90. The spinal implant of claim 87, wherein said internal chamber is capable of containing bone growth promoting material.

91. The spinal implant of claim 90, further comprising said bone growth promoting material, said bone growth promoting material being one of bone morphogenetic protein, hydroxyapatite, and genes coding for the production of bone.

92. The spinal implant of claim 57, further comprising at least one opening capable of retaining fusion promoting materials.

93. The spinal implant of claim 57, in combination with a fusion promoting substance.

94. The spinal implant of claim 93, wherein said fusion promoting substance includes at least one of bone, bone morphogenetic protein, hydroxyapatite, and genes coding for the production of bone.

95. The spinal implant of claim 57, wherein each of said first and second surface projections have a base, said bases being adjacent to one another.

96. The spinal implant of claim 57, wherein each of said first and second surface projections have a base, said bases being spaced apart from one another along a direction generally parallel to the mid-longitudinal axis of said implant.

97. The spinal implant of claim 57, wherein each of said first and second surface projections have a base, said bases being spaced apart from one another along a direction generally transverse to the mid-longitudinal axis of said implant.

98. The spinal implant of claim 57, wherein said forward facets of each of said first and second surface projections face the same direction.

99. The spinal implant of claim 57, wherein said opposed side facets are directed generally toward said spaced apart sides of said implant, respectively, said side facets converging toward each other in a direction away from one of said upper and lower surfaces of said implant.

100. The spinal implant of claim 57, wherein said opposed side facets intersect each other.

101. The spinal implant of claim 100, wherein said opposed side facets converge to form a peak at the top of said surface projection.

102. The spinal implant of claim 101, wherein said peaks of at least two of said surface projections are aligned along lines that are at least one of perpendicular, parallel, and diagonal to the mid-longitudinal axis of said implant.

103. The spinal implant of claim 101, wherein said peak of said first surface projection overlies at least a portion of said second surface projection.

104. The spinal implant of claim 101, wherein said peaks of said first and second surface projections are at the same height above one of said upper and lower surfaces of said implant.

105. The spinal implant of claim 99, wherein adjacent side facets of adjacent surface projections are spaced apart to define a groove therebetween.

106. The spinal implant of claim 99, wherein a plurality of adjacent surface projections are spaced apart to form a plurality of grooves therebetween.

107. The spinal implant of claim 106, wherein at least one of said grooves is parallel to the mid-longitudinal axis of said implant.

108. The spinal implant of claim 106, wherein at least two of said grooves cross each other.

109. The spinal implant of claim 106, wherein at least one of said grooves has a horizontal cross-sectional shape that is one of a v-shape, u-shape, and a box-like shape.

110. (canceled)

111. The spinal implant of claim 1, wherein said implant has a width and a height, the width of said implant being greater than the height.

112. The spinal implant of claim 1, wherein said forward facet, said rearward facet, and said opposed side facets each form a triangle.

113. The spinal implant of claim 1, wherein said upper and lower surfaces each include a plurality of openings to permit bone growth from adjacent vertebral body to adjacent vertebral body through said implant.

114. The spinal implant of claim 57, wherein said implant has a width and a height, the width of said implant being greater than the height.

115. The spinal implant of claim 57, wherein said forward facet, said rearward facet, and said opposed side facets each form a triangle.

116. The spinal implant of claim 57, wherein said upper and lower surfaces each include a plurality of openings to permit bone growth from adjacent vertebral body to adjacent vertebral body through said implant.

117. The spinal implant of claim 1, wherein said rear facet includes a perimeter having at least a first and a second portion arranged to form an apex having an included angle greater than 90 degrees between said first and second portions of the perimeter.

118. The spinal implant of claim 1, wherein said forward facet is directed only toward the leading end.

119. The spinal implant of claim 1, wherein said each of said opposed side facets have a surface area, said forward facet having a surface area larger than the surface area of each of said side facets.

120. The spinal implant of claim 1, wherein said at least one surface projection has a maximum height that is less than the length of said base.

121. The spinal implant of claim 57, wherein said rear facet includes a perimeter having at least a first portion and a second portion arranged to form an apex having an included angle greater than 90 degrees between said first and second portions of the perimeter.

122. The spinal implant of claim 57, wherein said forward facet is directed only toward the leading end.

123. The spinal implant of claim 57, wherein said each of said opposed side facets have a surface area, said forward facet having a surface area larger than the surface area of each of said side facets.

124. The spinal implant of claim 57, wherein said at least one surface projection has a maximum height that is less than the length of said base.
